(19) United States
(12) Patent Application Publication
(10) Pub. No.: US 2006/0226044 A1
(43) Pub. Date: Oct. 12, 2006

(54) Pacifier protective assembly

(76) Inventor: Naftali Lobl, Ramat Gan (IL)

Correspondence Address: Ezra Sutton & Associates, P.A., Plaza 9, 900 Route 9, Woodbridge, NJ, 07095, US

(21) Appl. No.: 11/101,694

(22) Filed: Apr. 8, 2005

(51) Int. Cl. A61B 19/02

(52) U.S. Cl. 206/438

(57) ABSTRACT

A protective assembly for receiving and protecting a pacifier, wherein the pacifier has a nipple and a plate for holding the nipple. The protective assembly includes a housing for receiving the nipple of the pacifier to protect it, a cover for receiving and holding the plate of the pacifier, and a pivot axle and biased spring connected between the cover and the housing for biasing the cover and the pacifier to automatically close and engage the housing with the nipple contained and protected within the housing. The cover has engaging means for engaging and holding the plate of the pacifier, so that the pacifier moves with the cover between open and closed positions relative to the housing so that in the closed position the housing receives and protects the nipple of the pacifier to keep it clean. Additionally, the cover has an opening and an inner surface and the handle of the pacifier passes through the opening, and the plate of the pacifier engages that inner surface of the cover; and wherein the cover has engaging means in the form of a bayonet locking device, wherein the pacifier is rotated relative to the cover to lock the pacifier nipple in its holding position within the housing.

### FIELD OF THE INVENTION

The present invention relates to a hinged pacifier protective assembly for receiving and protecting a pacifier such that when the pacifier accidentally falls out of a baby's mouth the hinged pacifier protective assembly protects the pacifier from hitting a floor and getting contaminated. More particularly, the hinged pacifier protective assembly includes a cover section and a housing section that automatically closes to protect the pacifier when not in use by a baby by means of a spring-biased hinge member.

### BACKGROUND OF THE INVENTION

Pacifier cover devices are well known in the art. These pacifier cover devices provide a means for hygienically protecting a pacifier when not in use. Typically, these devices do not completely keep the pacifier in a sanitized and/or hygienic condition when a baby discards their pacifier from his/or her mouth when not in use.

There remains a need for a hinged pacifier protective assembly that protects the pacifier when not in use by a baby, infant or child. More particularly, the hinged pacifier protective assembly will protect the pacifier when the pacifier accidentally falls out of an infant's or baby's mouth such that the pacifier is protected when hitting a floor and from being contaminated after hitting the floor. Additionally, the hinged pacifier protective assembly will have a cover section and a housing section that will automatically close by a spring-biased hinge member when not in use. The hinged pacifier protective assembly of the present invention has overcome the drawbacks of conventional sanitary pacifier covers and has an improved cover for a more convenient use and storage, as well as for preventing its contamination.

### Description of the Prior Art

Hygienic pacifier shields, pacifier covers, pacifier cases, hygienic pacifier containers and the like, having various designs, structures, configurations and materials of construction have been disclosed in the prior art. For example, U.S. Pat. No. 5,578,058 to CHEN discloses a hygienic pacifier shield with a bivalve shell. The hygienic pacifier shield includes a base member, a nipple protruding from the base member, a handle secured to the base member, and a bivalve shell hingedly secured to the base member. The bivalve shell has a pair of half-shell pieces separable and openable beyond a baby's mouth to reveal the nipple or pacifier position. The pair of half-shell pieces is foldably closeable for shielding the nipple portion with in the half-shell pieces for preventing contamination of the nipple portion. In addition the two half-shell pieces can be automatically closed by a restoring spring at the hinges of the shell for automatically restoring the two half-shell pieces to a closed position to always keep the nipple portion clean when not used for pacifying a baby. This prior art patent does not disclose or teach the structure and configuration of the hinged pacifier protective assembly of the present invention.

U.S. Pat. No. 5,156,617 to REID discloses a pacifier cover having a single shell or cup-shaped cover for keeping a pacifier clean. The cover device includes a cover which is hingeably attached to the base, and snaps for removably securing the pacifier to the base. The cover is removable between a closed position in which the cover encloses the pacifier and prevents contact with the surrounding environment, and an open position in which the pacifier may be attached to or removed from the base. This prior art patent does not disclose or teach the structure and configuration of the hinged pacifier protective assembly of the present invention.

U.S. Pat. No. 5,722,537 to SIGLER discloses a portable disinfectant container for an infant pacifier or infant nursing nipple, consisting of a half-oval shaped disinfectant container which is provided with a sponge that may be saturated with any appropriate non-toxic disinfectant solution. The disinfectant container is constructed of durable ABS plastic and has an attached hook for hanging on a purse, baby bag, stroller, or crib. The hinged lid of the disinfectant container may be flipped open and a pacifier or nipple inserted into a slit in the sponge for disinfectant purposes, the absorption qualities of the sponge preventing any type of spillage of the disinfectant. This prior art patent does not disclose or teach the structure and configuration of the hinged pacifier protective assembly of the present invention.

None of these prior art patents teach or disclose the structure and configuration of a hinged pacifier protective assembly having a cover section and a housing section being pivotally connected by a spring-biased hinge member for automatically closing the cover section and the housing section together when not in use by an infant.

Accordingly, it is an object of the present invention to provide a hinged pacifier protective assembly that hygienically protects a pacifier when not in use by an infant, baby or child.

Another object of the present invention is to provide a hinged pacifier protective assembly that protects the pacifier when the pacifier accidentally falls out of the infant's mouth and such that the pacifier is hygienically protected when hitting a floor and from being contaminated.

Another object of the present invention is to provide a hinged pacifier protective assembly that includes a cover section and a housing section being pivotally connected by a spring-biased hinge member for automatically closing each of the cover and housing sections together when not in use by the infant.

Another object of the present invention is to provide a hinged pacifier protective assembly that is made of flexible and durable plastic materials for easy cleaning and sanitizing for multiple re-use by a parent for their children's safety.

Another object of the present invention is to provide a hinged pacifier protective assembly that conveniently stores the pacifier within the closed cover and housing sections when not in use and prevents the pacifier from any contamination when in the closed configuration.

A further object of the present invention is to provide a hinged pacifier protective assembly that can be mass produced in an automated and economical manner and is readily affordable by parents.

### SUMMARY OF THE INVENTION

In accordance with the present invention, there is provided a protective assembly for receiving and protecting a pacifier, wherein the pacifier has a nipple and a plate for holding the nipple. The protective assembly includes a housing for receiving the nipple of the pacifier to protect it, a cover for receiving and holding the plate of the pacifier, and a pivot axle and biased spring connected between the cover and the housing for biasing the cover and the pacifier to automatically close and engage the housing with the nipple contained and protected within the housing. The cover has engaging means for engaging and holding the plate of the pacifier, so that the pacifier moves with the cover between open and closed positions relative to the housing so that in the closed position the housing receives and protects the nipple of the pacifier to keep it clean. Additionally, the cover has an opening and an inner surface and the handle of the pacifier passes through the opening, and the plate of the pacifier engages the inner surface of the cover, and wherein the cover has engaging means in the form of a bayonet locking device, wherein the pacifier is rotated relative to the cover to lock the pacifier nipple in its holding position within the housing.

### BRIEF DESCRIPTION THE OF DRAWINGS

Further objects, features and advantages of the present invention will become apparent upon the consideration of the following detailed description of the presently preferred embodiment when taken in conjunction with the accompanying drawings, wherein:

FIG. 1 is a partially exploded perspective view of the pacifier protective assembly of the first embodiment of the present invention showing the major component parts thereof and in an opened configuration being readied for operational use thereof;

FIG. 2 is a perspective view of the pacifier protective assembly of the first embodiment of the present invention showing the protective assembly in a closed configuration;

FIG. 3 is a partially exploded perspective view of the pacifier protective assembly of the second embodiment of the present invention showing the major component parts thereof and in an opened configuration being readied for operational use thereof;

FIG. 4 is a perspective view of the pacifier protective assembly of the second embodiment of the present invention showing the protective assembly in an assembled state and in an opened configuration;

FIG. 5 is a partially exploded perspective view of the pacifier protective assembly of the third embodiment of the present invention showing the major component parts thereof and in an opened configuration being readied for operational use thereof;

FIG. 6 is a perspective view of the pacifier protective assembly of the third embodiment of the present invention showing the protective assembly in an assembled state and in an opened configuration.

FIG. 7 is a partially exploded perspective view of the pacifier protective assembly of the fourth embodiment of the present invention showing the major component parts thereof and in an opened configuration being readied for operational use thereof;

FIG. 8 is a perspective view of the pacifier protective assembly of the fourth embodiment of the present invention showing the protective assembly in an assembled state and in an opened configuration;

FIG. 9 is a partial cross-sectional view of the pacifier protective assembly of the fourth embodiment of the present invention showing engaging means in the form of threads within a compartment opening of a cover;

FIG. 10 is a partial cross-sectional view of the pacifier protective assembly of the fourth embodiment of the present invention showing the engaging means in the form of threads within the compartment opening of the cover;

FIG. 11 is a partially exploded perspective view of the pacifier protective assembly of the fifth embodiment of the present invention showing the major component parts thereof and in an opened configuration being readied for operational use thereof;

FIG. 12 is a perspective view of the pacifier protective assembly of the fifth embodiment of the present invention showing a pair of clamping sections being pivotally moved in an inwardly direction for locking a plate of a pacifier nipple in a holding position within a housing;

FIG. 13 is a perspective view of the pacifier protective assembly of the fifth embodiment of the present invention showing the protective assembly in an assembled state, in an opened configuration and the clamping sections in a closed configuration about the plate of the pacifier nipple;

FIG. 14 is a partially exploded perspective view of the pacifier protective assembly of the sixth embodiment of the present invention showing the major component parts thereof and in an opened configuration being readied for operational use thereof;

FIG. 15 is a perspective view of the pacifier protective assembly of the sixth embodiment of the present invention showing the protective assembly in an assembled state and in an opened configuration;

FIG. 16 is a partially exploded perspective view of the pacifier protective assembly of the seventh embodiment of the present invention showing the major component parts thereof and in an opened configuration being readied for operational use thereof;

FIG. 16A is a perspective view of the pacifier protective assembly of the seventh embodiment of the present invention showing engaging means in the form of a snap;

FIG. 17 is a perspective view of the pacifier protective assembly of the seventh embodiment of the present invention showing the protective assembly in an assembled state and in a opened configuration;

FIG. 18 is a partially exploded perspective view of the pacifier protective assembly of the eighth embodiment of the present invention showing the major component parts thereof and in opened configuration being readied for operational use thereof;

FIG. 18A is a perspective view of the pacifier protective assembly of the eighth embodiment of the present invention showing engaging means in the form of a suction cup;

FIG. 19 is a partially exploded perspective of the pacifier protective assembly of the ninth embodiment of the present invention showing the major component parts thereof and in an opened configuration being readied for operational use thereof;

FIG. 20 is a perspective view of the pacifier protective assembly of the ninth embodiment of the present invention showing the protective assembly in an assembled state and in an opened configuration;

FIG. 20A is a perspective view of the pacifier protective assembly of the ninth embodiment of the present invention showing engaging means in the form of a locking element received within mounting openings within the cover and the plate of the pacifier;

FIG. 21 is a perspective view of the pacifier protective assembly of the tenth embodiment of the present invention showing the protective assembly in an assembled state and in an opened configuration;

FIG. 22 is a perspective view of the pacifier protective assembly of the tenth embodiment of the present invention showing the protective assembly in a closed configuration and engaging means in the form of a sliding locking latch;

FIG. 23 is a perspective view of the pacifier protective assembly of the eleventh embodiment of the present invention showing an exterior wall surface of the plate of the pacifier without a nipple having a plurality of loop fastener strips thereon;

FIG. 24 is a partially exploded perspective view of the pacifier protective assembly of the eleventh embodiment of the present invention showing the major component parts thereof and in an opened configuration being readied for operational use thereof;

FIG. 25 is a perspective view of the pacifier protective assembly of the twelfth embodiment of the present invention showing the protective assembly in an assembled state and in an opened configuration;

FIG. 26 is a perspective view of the pacifier protective assembly of the twelfth embodiment of the present invention showing the protective assembly in a closed configuration;

FIG. 27 is an exploded perspective view of the pacifier protective assembly of the thirteenth embodiment of the present invention showing a pacifier engaging a slot opening for inserting the pacifier within a cover member in an opened configuration being readied for operational use thereof;

FIG. 27A is an enlarged perspective view of the pacifier protective assembly of the thirteenth embodiment of the present invention showing a snapping element for engaging the handle of the pacifier;

FIG. 28 is a sectional view of the pacifier protective assembly of the thirteenth embodiment of the present invention showing the pacifier in a locked state and the protective assembly in a closed configuration;

FIG. 29 is a perspective view of the pacifier protective assembly of the thirteenth embodiment of the present invention showing the protective assembly in an assembled state and in an opened configuration; and

FIG. 29A is an enlarged perspective view of the pacifier protective assembly of the thirteenth embodiment of the present invention showing the snapping element engaged with the handle of the pacifier.

### DETAILED DESCRIPTION OF THE EMBODIMENTS

### First Embodiment 100

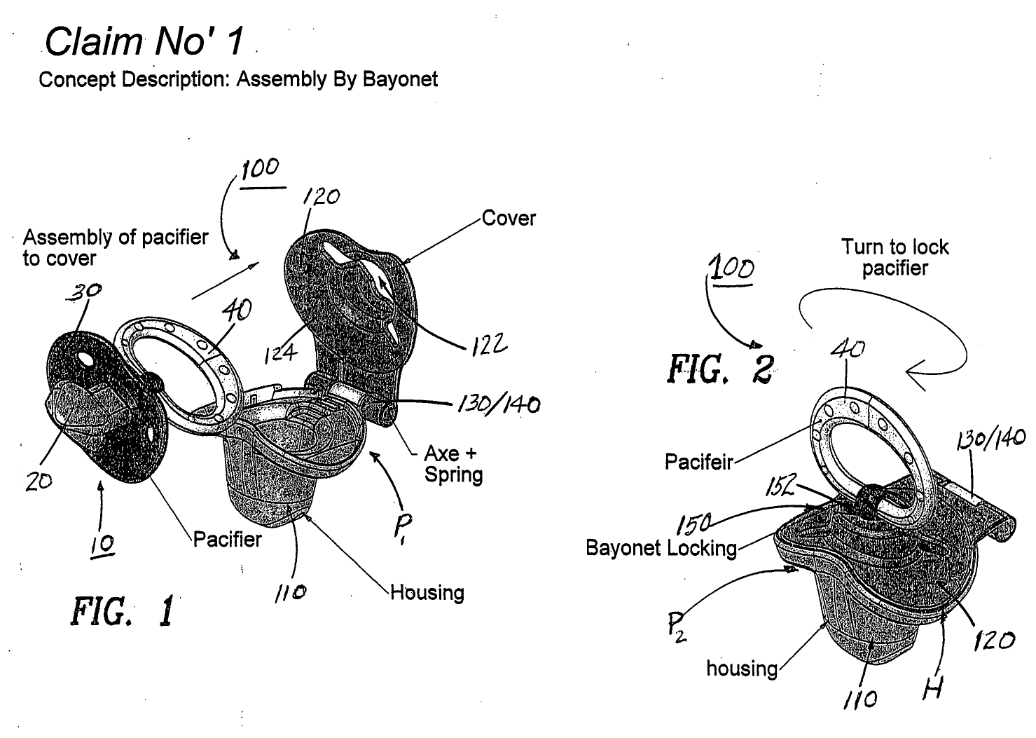

A protective assembly 100 for receiving and protecting a pacifier 10, as shown in FIGS. 1 and 2, wherein the pacifier 10 has a nipple 20 and a plate 30 for holding the nipple 20. The protective assembly 100 includes a housing 110 for receiving the nipple 20 of the pacifier 10, to protect it, a cover 120 for receiving and holding the plate 30 of the pacifier 10, and a pivot axle 130 and biased spring 140 connected between the cover 120 and the housing 110 for biasing the cover 120 and the pacifier 10 to automatically close and engage the housing 110, with the nipple 20 contained and protected within the housing 110. Also, the cover 120 has engaging means 150 for engaging and holding the plate 30 of the pacifier 10, so that the pacifier 10 moves with the cover 120 between open and closed positions P₁ and P₂ relative to the housing 110 so that in the closed position P₂ the housing 110 receives and protects the nipple 20 of the pacifier 10 to keep it clean.

Additionally, the cover 120 has an opening 122 and an inner surface 124 and the handle 40 of the pacifier 10 passes through the opening 122 and the plate 30 of the pacifier 10 engages the inner surface 124 of the cover 120; and wherein the cover 120 has engaging means 150 in the form of a bayonet locking device 152, wherein the pacifier 10 is rotated relative to the cover 120 to lock the pacifier nipple 20 in its holding position H within the housing 110.

### Second Embodiment 200

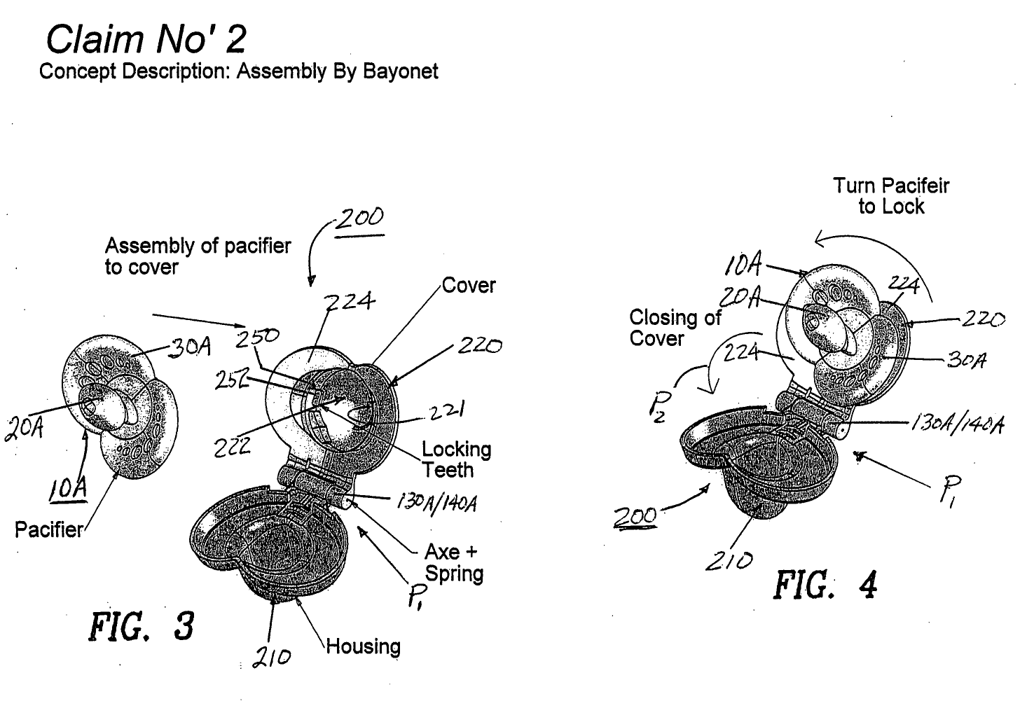

A protective assembly 200 for receiving and protecting a pacifier 10A, as shown in FIGS. 3 and 4, wherein the pacifier 10A has a nipple 20A and a plate 30A for holding the nipple 20A. The protective assembly 200 includes a housing 210 for receiving the nipple 20A of the pacifier 10A, to protect it, a cover 220 for receiving and holding the plate 30A of the pacifier 10, and a pivot axle 130A and biased spring 140A connected between the cover 220 and the housing 210 for biasing the cover 220 and the pacifier 10A to automatically close and engage the housing 210, with the nipple 20A contained and protected within the housing 210. Also, the cover 220 has engaging means 250 for engaging and holding the plate 30A of the pacifier 10A, so that the pacifier 10A moves with the cover 220 between open and closed positions P₁ and P₂ relative to the housing 210 so that in the closed position P₂ the housing 210 receives and protects the nipple 20A of the pacifier 10A to keep it clean.

Additionally, the 220 cover has a compartment 221 and opening 222 and an inner surface 224, and the plate 30A of the pacifier 10A engages the inner surface 224 of the cover 220; and wherein the 220 has engaging means 250 in the form of locking teeth 252, wherein the pacifier 10A is rotated within the compartment opening 222 relative to the cover 220 to lock the pacifier nipple 20A in its holding position H within the housing 210.

### Third Embodiment 300

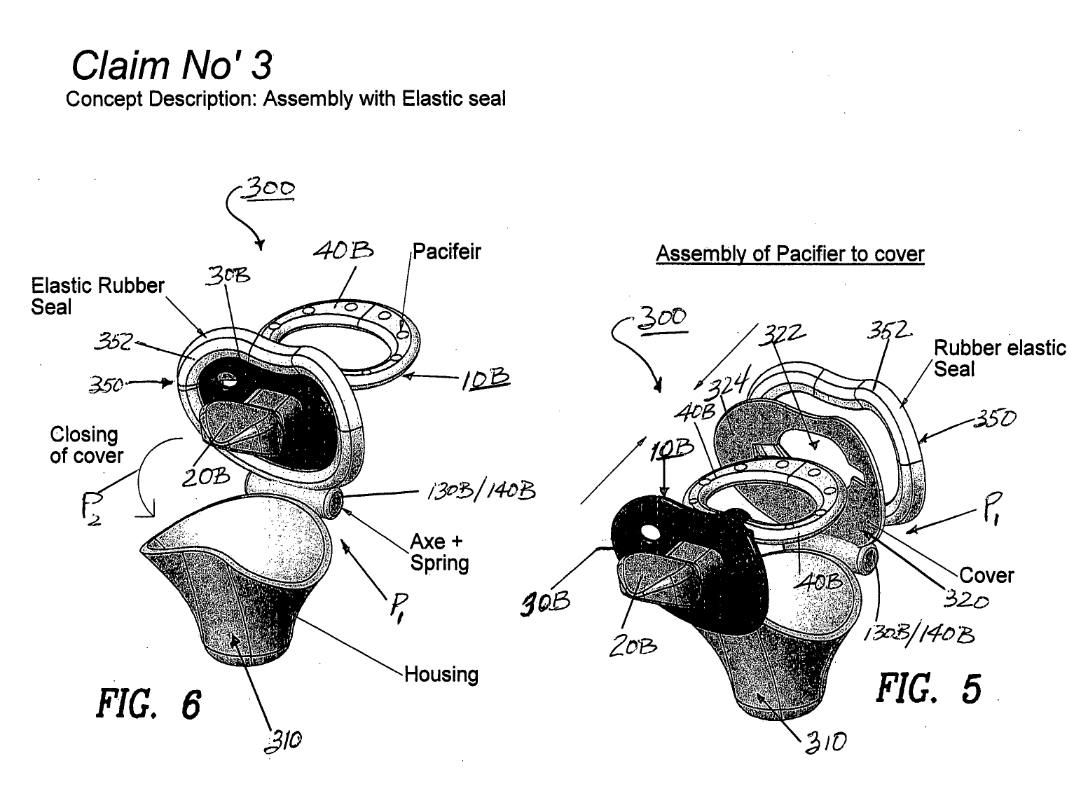

A protective assembly 300 for receiving and protecting a pacifier 10B, as shown in FIGS. 5 and 6, wherein the pacifier 10B has a nipple 20B and a plate 30B for holding the nipple 20B. The protective assembly 300 includes a housing 310 for receiving the nipple 20B of the pacifier 10B, to protect it, a cover 320 for receiving and holding the plate 30B of the pacifier 10B, and a pivot axle 130B and biased spring 140B connected between the cover 320 and the housing 310 for biasing the cover 320 and the pacifier 10B to automatically close and engage the housing 310, with the nipple 20B contained and protected within the housing 310. Also, the cover 320 has engaging means 350 for engaging and holding the plate 30B of the pacifier 10B, so that the pacifier 10B moves with the cover 320 between open and closed positions P₁ and P₂ relative to the housing 310 so that in the closed position P₂ the housing 310 receives and protects the nipple 20B of the pacifier 10B to keep it clean.

Additionally, the 320 cover has a compartment opening 322 and an inner surface 324, and the handle 40B of the pacifier 10B passes through the openings 322, and the plate 30B of the pacifier 10B engages the inner surface 324 of the cover 320, and wherein the cover 320 has engaging means 350 in the form of a rubber elastic seal 352 for engaging and sealing the cover 320 and the plate 30B of the pacifier 10B within the rubber elastic seal 352; wherein the rubber elastic seal 352 locks the cover 320 of the pacifier nipple 20B in its holding position H within the housing 310.

### Fourth Embodiment 400

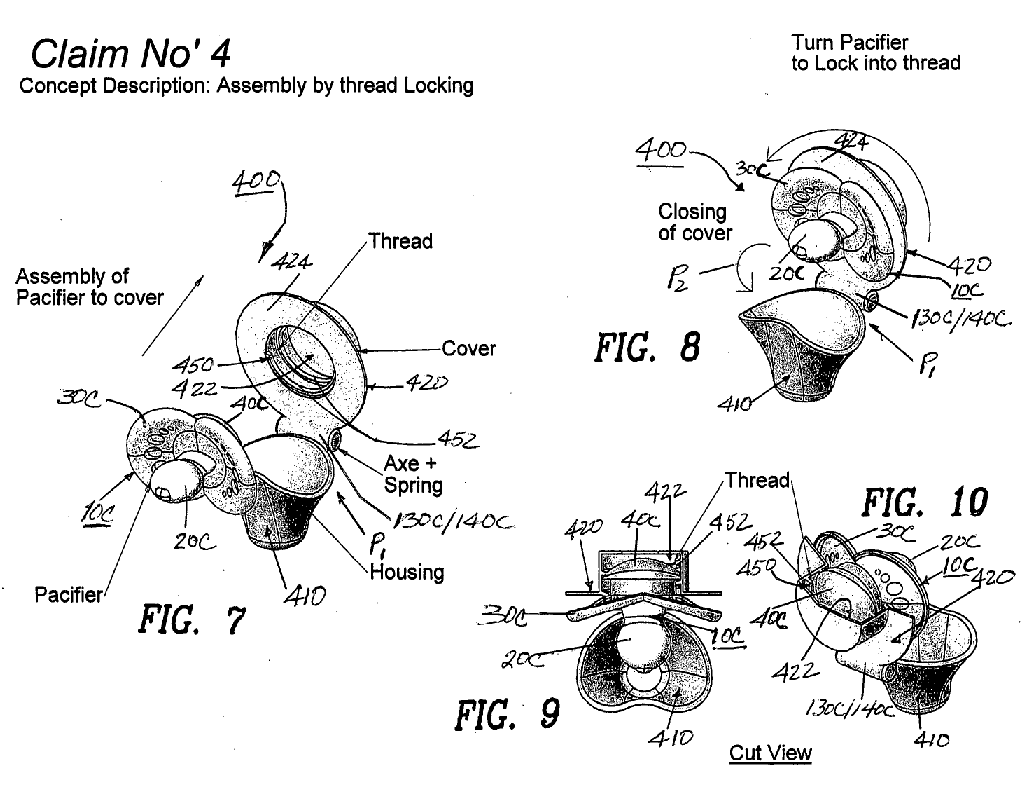

A protective assembly 400 for receiving and protecting a pacifier 10C, as shown in FIGS. 7 to 10, wherein the pacifier 10C has a nipple 20C and a plate 30C for holding the nipple 20C. The protective assembly 400 includes a housing 410 for receiving the nipple 20C of the pacifier 10C, to protect it, a cover 420 for receiving and holding the plate 30C of the pacifier 10C, and a pivot axle 130C and biased spring 140C connected between the cover 420 and the housing 410 for biasing the cover 420 and the pacifier 10C to automatically close and engage the housing 410, with the nipple 20C contained and protected within the housing 410. Also, the cover 420 has engaging means 450 for engaging and holding the plate 30C of the pacifier 10C, so that the pacifier 10C moves with the cover 420 between open and closed positions P₁ and P₂ relative to the housing 410 so that in the closed position P₂ the housing 410 receives and protects the nipple 20C of the pacifier 10C to keep it clean.

Additionally, the cover 420 has a compartment opening 422 and an inner surface 424, and the handle 40C of the pacifier 10C is positioned within the compartment opening 422 of the cover 420, and the plate 30C of the pacifier 10C engages the inner surface 424 of the cover 420; and wherein the cover 420 has engaging means 450 in the form of threads 452, and wherein the handle 40C of the pacifier 10C is rotated relative to the cover 420 to lock the pacifier nipple 20C in its holding position H within the housing 410.

### Fifth Embodiment 500

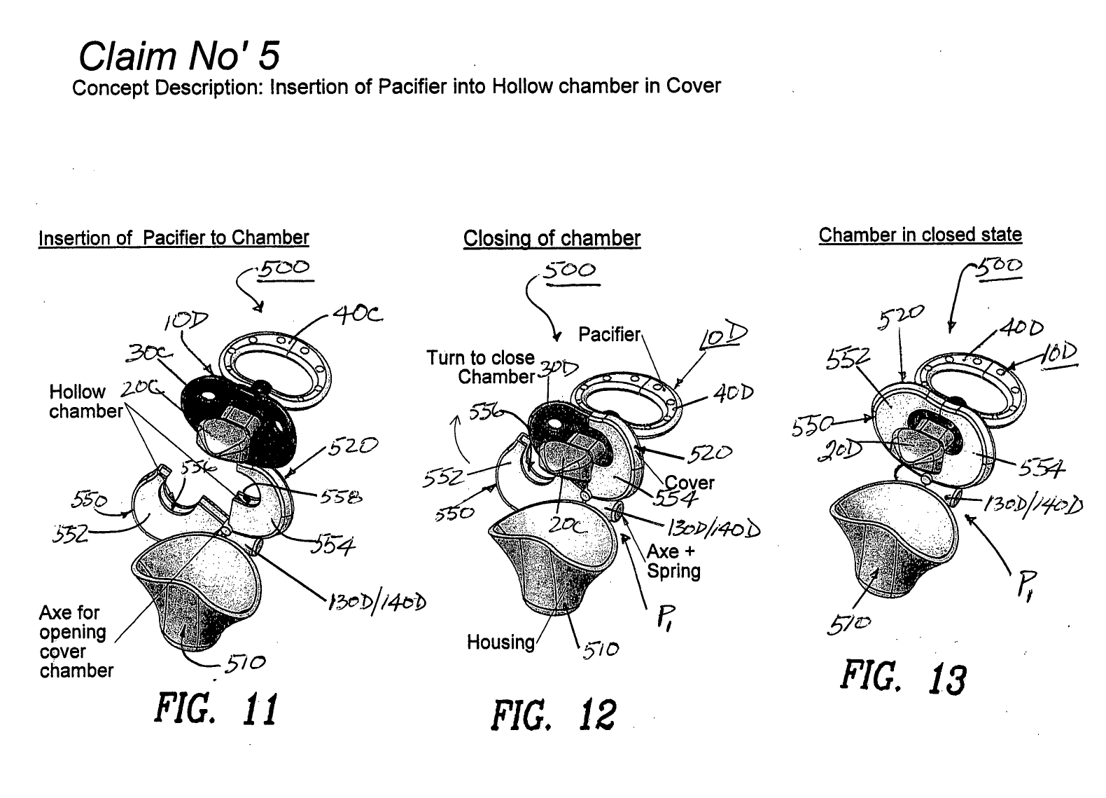

A protective assembly 500 for receiving and protecting a pacifier 10D, as shown in FIGS. 11 to 13, wherein the pacifier 10D has a nipple 20D and a plate 30D for holding the nipple 20D. The protective assembly 500 includes a housing 510 for receiving the nipple 20D of the pacifier 10D, to protect it, a cover 520 for receiving and holding the plate 30D of the pacifier 10D, and a pivot axle 130D and biased spring 140D connected between the cover 520 and the housing 510 for biasing the cover 520 and the pacifier 10D to automatically close and engage the housing 510, with the nipple 20D contained and protected within the housing 510. Also, the cover 520 has engaging means 550 for engaging and holding the plate 30D of the pacifier 10D, so that the pacifier 10D moves with the cover 520 between open and closed positions P₁ and P₂ relative to the housing 510 so that in the closed position P₂ the housing 510 receives and protects the nipple 20D of the pacifier 10D to keep it clean.

Additionally, the cover 520 has first and second clamping sections 552 and 554 each having an interior opening 556 and 558, respectively, and the plate 30D of the pacifier 10D engages each of the interior openings 556 and 558 of the first and second clamping sections 552 and 554; and wherein the cover 520 has engaging means 550 in the form of the clamping sections 552 and 554, wherein each of the clamping sections 552 and 554 are pivotally moved in an inwardly direction in order to lock the plate 30D and the pacifier nipple 20D in its holding position H within the housing 510.

### Sixth Embodiment 600

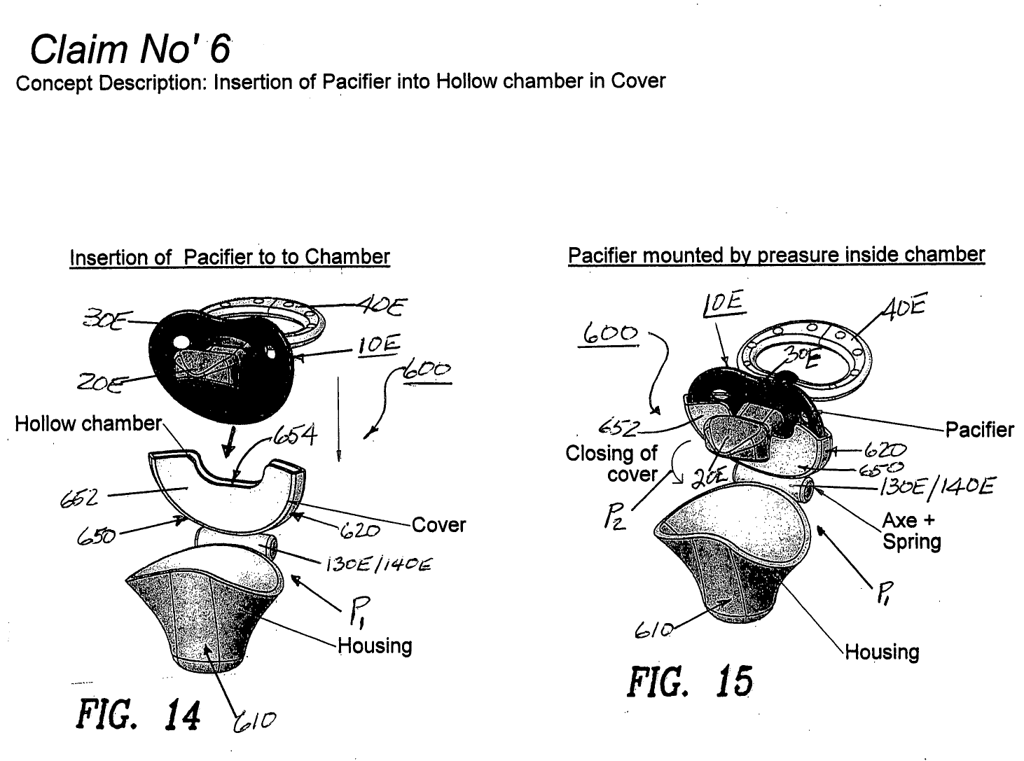

A protective assembly 600 for receiving and protecting a pacifier 10E, as shown in FIGS. 14 and 15, wherein the pacifier 10E has a nipple 20E and a plate 30E for holding the nipple 20E. The protective assembly 600 includes a housing 610 for receiving the nipple 20E of the pacifier 10E, to protect it, a cover 620 for receiving and holding the plate 30E of the pacifier 10E, and a pivot axle 130E and biased spring 140E connected between the cover 620 and the housing 610 for biasing the cover 620 and the pacifier 10E to automatically close and engage the housing 610, with the nipple 20D contained and protected within the housing 610. Also, the cover 620 has engaging means 650 for engaging and holding the plate 30E of the pacifier 10E, so that the pacifier 10E moves with the cover 620 between open and closed positions P₁ and P₂ relative to the housing 610 so that in the closed position P₂ the housing 610 receives and protects the nipple 20E of the pacifier 10E to keep it clean.

Additionally, the cover 620 has a chamber section 652 having an interior channel opening 654 therein such that a portion of the plate 30E engages the interior channel opening 654 of the chamber section 652; and wherein the cover 620 has engaging means 650 in the form of the chamber section 652, wherein a portion of plate 30E is slidably received within the interior channel opening 654 of the chamber section 652 in order to lock a portion of plate 30E of the pacifier nipple 20E in its holding position H within the housing 610.

### Seventh Embodiment 700

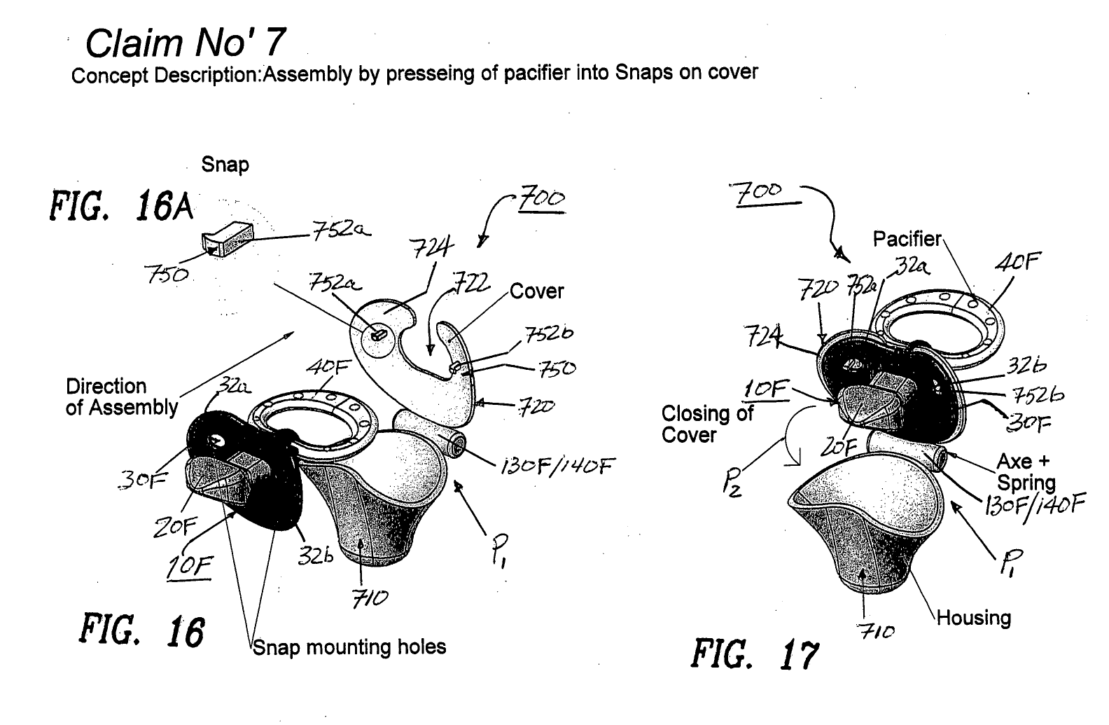

A protective assembly 700 for receiving and protecting a pacifier 10F, as shown in FIGS. 16, 16A and 17, wherein the pacifier 10F has a nipple 20F and a plate 30F for holding the nipple 20F. The protective assembly 700 includes a housing 710 for receiving the nipple 20F of the pacifier 10F, to protect it, a cover 720 for receiving and holding the plate 30F of the pacifier 10F, and a pivot axle 130F and biased spring 140F connected between the cover 720 and the housing 710 for biasing the cover 720 and the pacifier 10F to automatically close and engage the housing 710, with the nipple 20F contained and protected within the housing 710. Also, the cover 720 has engaging means 750 for engaging and holding the plate 30F of the pacifier 10F, so that the pacifier 10F moves with the cover 720 between open and closed positions P₁ and P₂ relative to the housing 710 so that in the closed position P₂ the housing 710 receives and protects the nipple 20F of the pacifier 10F to keep it clean.

Additionally, the cover 720 has an opening 722 and an inner surface 724, the plate 30F has a pair of opposing and spaced-apart snap mounting openings 32a and 32b and the handle 40F of the pacifier 10F passes through the opening 722 and the plate 30F of the pacifier 10F engages the inner surface 722 of the cover 720; and wherein the cover 720 has engaging means 750 in the form of a pair of opposing and spaced-apart snaps 752a and 752b on the inner surface 722 of the cover 720 for detachably engaging the opposing and spaced-apart mounting openings 32a and 32b of the plate 30F in order to lock and hold the plate 30F and the pacifier nipple 20F in its holding position H within the housing 710.

### Eighth Embodiment 800

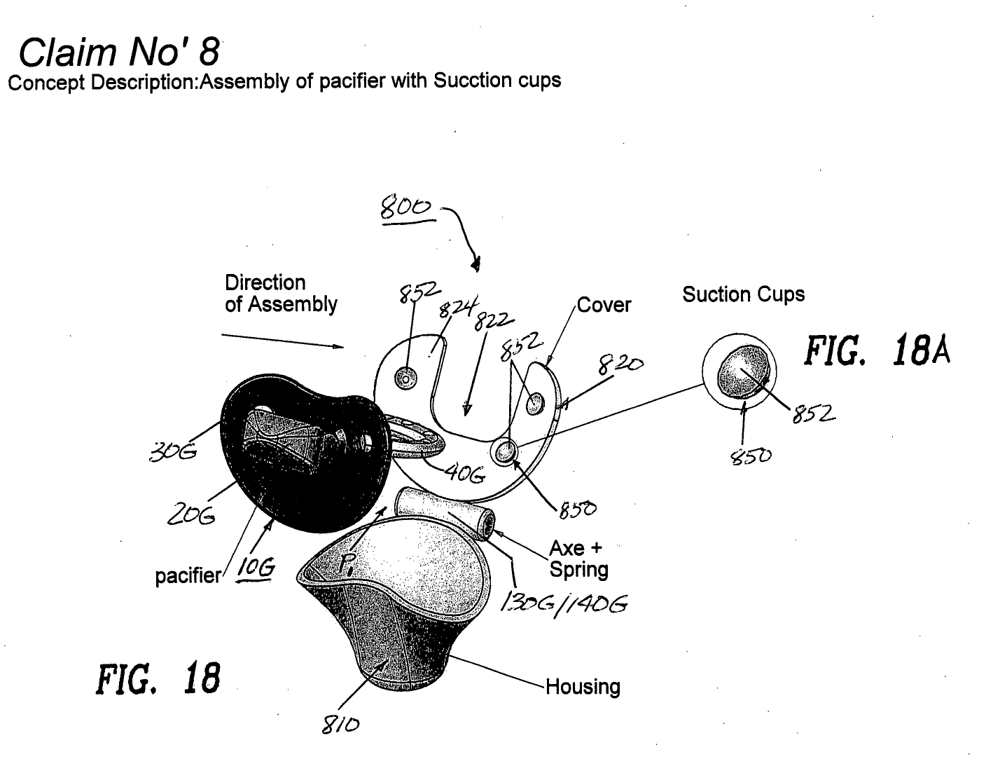

A protective assembly 800 for receiving and protecting a pacifier 10G, as shown in FIGS. 18 and 18A, wherein the pacifier 10G has a nipple 20G and a plate 30G for holding the nipple 20G. The protective assembly 800 includes a housing 810 for receiving the nipple 20G of the pacifier 10G, to protect it, a cover 820 for receiving and holding the plate 30G of the pacifier 10G, and a pivot axle 130G and biased spring 140G connected between the cover 820 and the housing 810 for biasing the cover 820 and the pacifier 10G to automatically close and engage the housing 810, with the nipple 20G contained and protected within the housing 810. Also, the cover 820 has engaging means 850 for engaging and holding the plate 30G of the pacifier 10G, so that the pacifier 10G moves with the cover 820 between open and closed positions P₁ and P₂ relative to the housing 810 so that in the closed position P₂ the housing 810 receives and protects the nipple 20G of the pacifier 10G to keep it clean.

Additionally, the cover 820 has a U-shaped opening 822 and an inner surface 824 and the handle 40G of the pacifier 10G passes through the U-shaped opening 822, and the plate 30G of the pacifier 10G engages the inner surface 824 of the cover 820; and wherein the cover 820 has engaging means 850 in the form of a plurality of suction cups 852 on the inner surface 824 of the cover 820, wherein the plate 30G detachably connects to the plurality of suction cups 852 in order to lock and hold the plate 30G and the pacifier nipple 20G in its holding position H within the housing 810.

### Ninth Embodiment 900

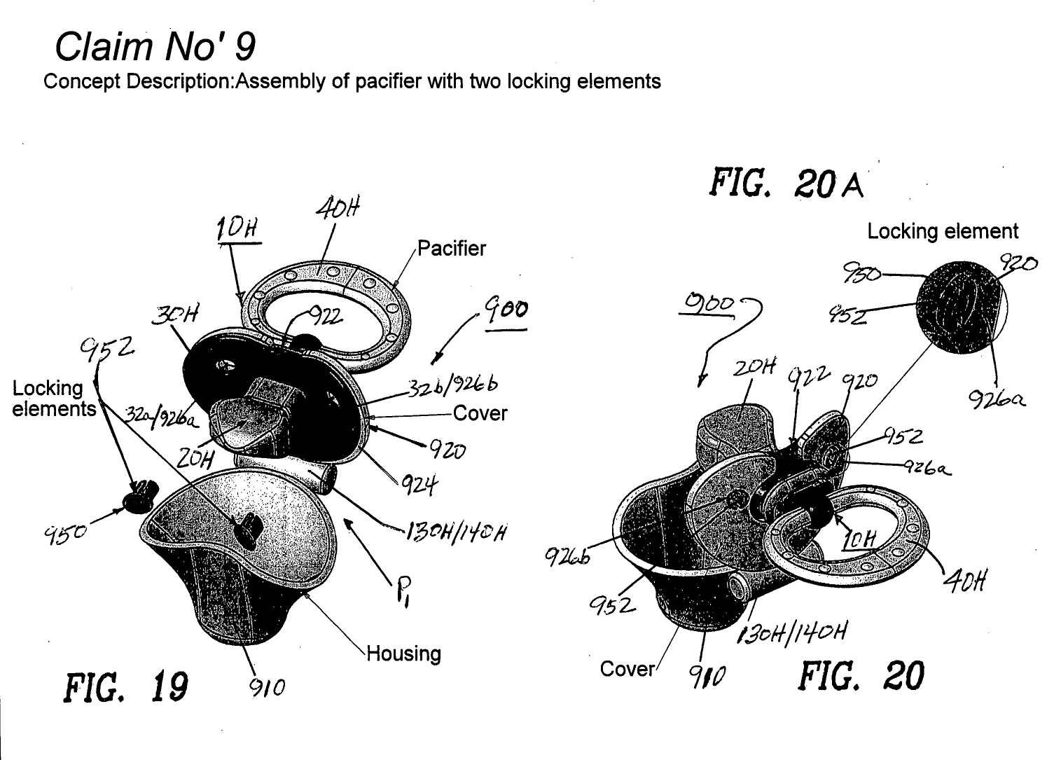

A protective assembly 900 for receiving and protecting a pacifier 10H, as shown in FIGS. 19, 20 and 20A, wherein the pacifier 10H has a nipple 20H and a plate 30H for holding the nipple 20H. The protective assembly 900 includes a housing 910 for receiving the nipple 20H of the pacifier 10H, to protect it, a cover 920 for receiving and holding the plate 30H of the pacifier 10H, and a pivot axle 130H and biased spring 140H connected between the cover 920 and the housing 910 for biasing the cover 920 and the pacifier 10H to automatically close and engage the housing 910, with the nipple 20H contained and protected within the housing 910. Also, the cover 920 has engaging means 950 for engaging and holding the plate 30H of the pacifier 10H, so that the pacifier 10H moves with the cover 920 between open and closed positions P₁ and P₂ relative to the housing 910 so that in the closed position P₂ the housing 910 receives and protects the nipple 20H of the pacifier 10H to keep it clean.

Additionally, the cover 920 has an opening 922, an inner surface 924 and a pair of opposing and spaced-apart mounting openings 926a and 926b, and the plate 30H has a pair of opposing and spaced-apart locking openings 32a and 32b, and the handle 40H of the pacifier 10H passes through the opening 922, and the plate 30H of the pacifier 10H engages the inner surface 924 of the cover 920 such that the locking openings 32a and 32b and the mounting openings 926a and 926b are aligned with each other on the plate 30H and the cover, respectively; and wherein the cover 920 has engaging means 950 in the form of one or more locking elements 952 for detachably engaging the aligned locking openings 32a and 32b and the mounting openings 926a and 926b, respectively, in order to lock and hold the plate 30H and the pacifier nipple 20H in its holding position within the housing 910.

### Tenth Embodiment 1000

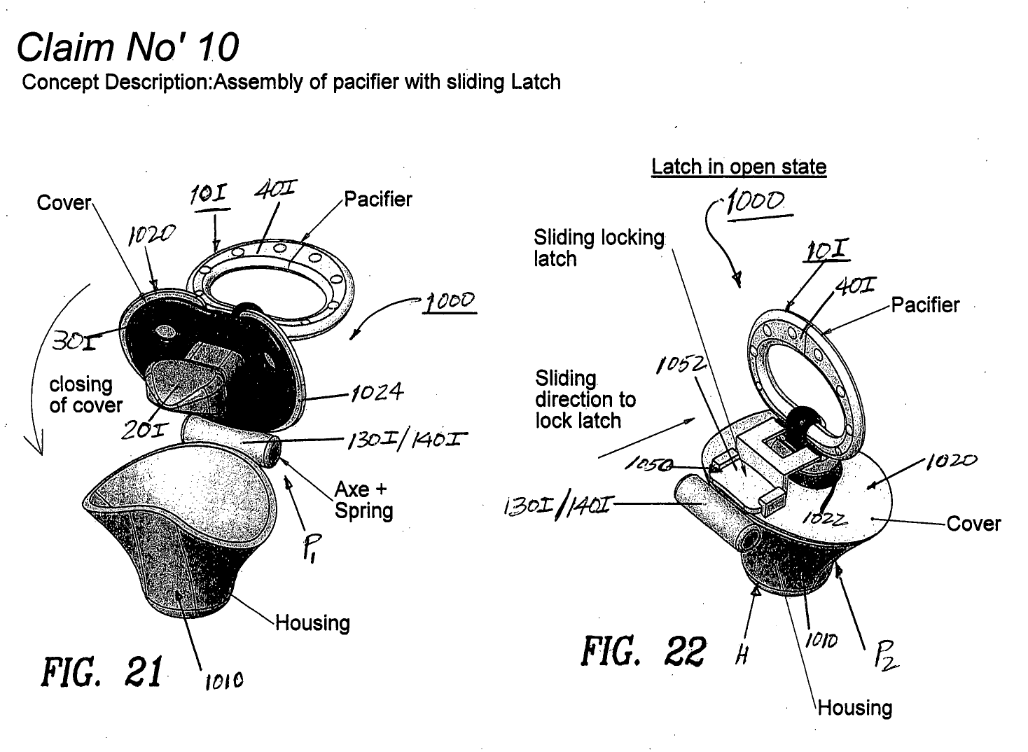

A protective assembly 1000 for receiving and protecting a pacifier 10I, as shown in FIGS. 21 and 22, wherein the pacifier 10I has a nipple 20I and a plate 30I for holding the nipple 20I. The protective assembly 1000 includes a housing 1010 for receiving the nipple 20I of the pacifier 10I, to protect it, a cover 1020 for receiving and holding the plate 30I of the pacifier 10I, and a pivot axle 130I and biased spring 140I connected between the cover 1020 and the housing 1010 for biasing the cover 1020 and the pacifier 10I to automatically close and engage the housing 1010, with the nipple 20I contained and protected within the housing 1010. Also, the cover 1020 has engaging means 1050 for engaging and holding the plate 30I of the pacifier 10I, so that the pacifier 10I moves with the cover 1020 between open and closed positions P₁ and P₂ relative to the housing 1010 so that in the closed position P₂ the housing 1010 receives and protects the nipple 20I of the pacifier 10I to keep it clean.

Accordingly, the cover 1020 has an opening 1022 and an inner surface 1024 and the handle 40I of the pacifier 10I passes through the opening 1022, and the plate 30I of the pacifier 10I engages the inner surface 1024 of the cover 1020; and wherein the cover 1020 has engaging means 1050 in the form of a sliding locking latch 1052 for detachably engaging the cover 1020 and a portion of the handle 40I in order to lock and hold the plate 30I and the pacifier nipple 20I in its holding position H within the housing 1010.

### Eleventh Embodiment 1100

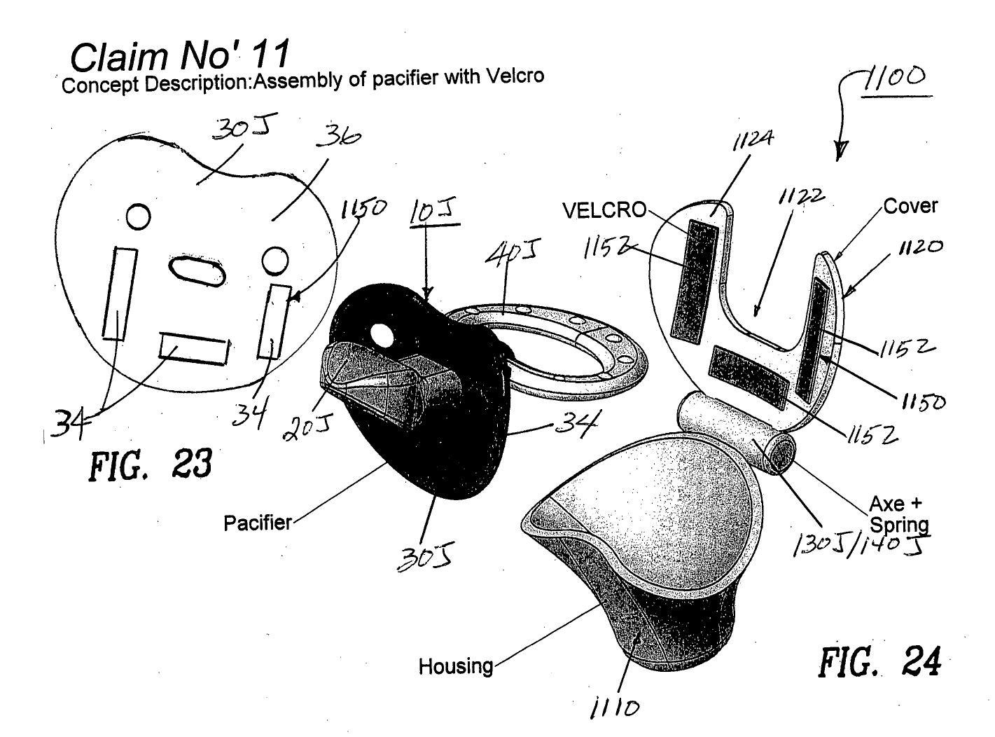

A protective assembly 1100 for receiving and protecting a pacifier 10J, as shown in FIGS. 23 and 24, wherein the pacifier 10J has a nipple 20J and a plate 30J for holding the nipple 20J. The protective assembly 1100 includes a housing 1110 for receiving the nipple 20J of the pacifier 10J, to protect it, a cover 1120 for receiving and holding the plate 30J of the pacifier 10J, and a pivot axle 130J and biased spring 140J connected between the cover 1120 and the housing 1110 for biasing the cover 1120 and the pacifier 10J to automatically close and engage the housing 1110, with the nipple 20J contained and protected within the housing 1110. Also, the cover 1120 has engaging means 1150 for engaging and holding the plate 30J of the pacifier 10J, so that the pacifier 10J moves with the cover 1120 between open and closed positions P₁ and P₂ relative to the housing 1110 so that in the closed position P₂ the housing 1110 receives and protects the nipple 20J of the pacifier 10J to keep it clean.

Additionally, the cover 1120 has a U-shaped opening 1122 and an inner surface 1124 and the handle 40J of the pacifier 10J passes through the U-shaped opening 1122, and the plate 30J has a plurality of loop fastener strips 34 (Velcro) positioned on an exterior surface 36 of the plate 30J; and wherein the cover 1120 has engaging means 1150 in the form of a plurality of hook fastener strips 1152 (Velcro) being positioned on the inner surface 1124 of the cover 1120, wherein the loop fastener strips 34 on the exterior surface 36 of the plate 30J are matched and aligned with the hook fastener strips 1152 on the inner surface 1124 of the cover 1120 in order to lock and hold the plate 30J and the pacifier nipple 20J in its holding position within the housing 1110.

### Twelfth Embodiment 1200

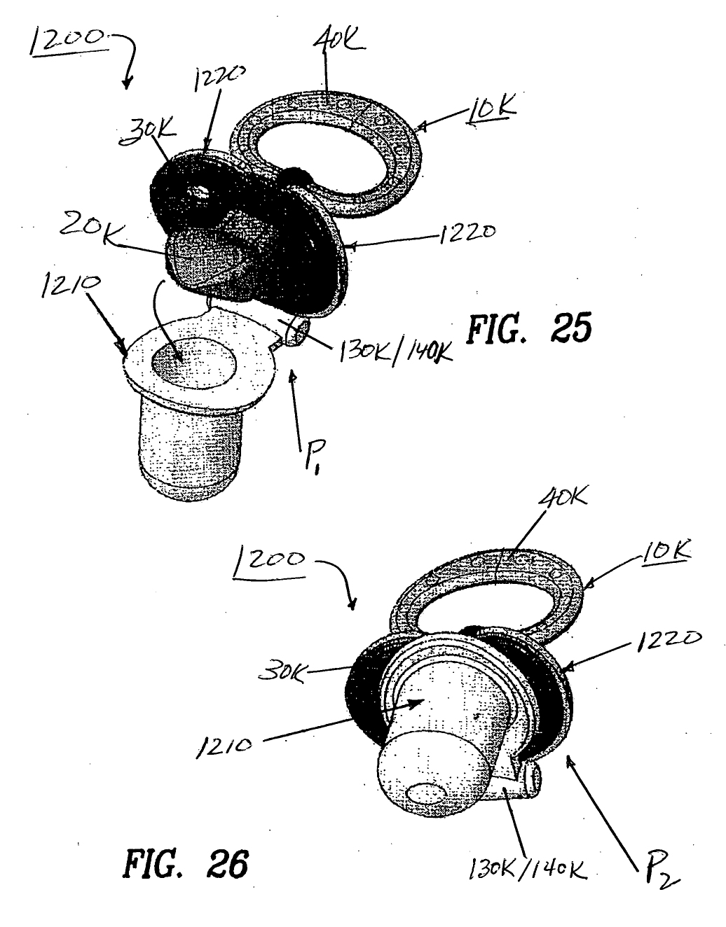

A protective assembly 1200 for receiving and protecting a pacifier 10K, as shown in FIGS. 25 and 26, wherein the pacifier 10K has a nipple 20K and a plate 30K for holding the nipple 20K. The protective assembly 1200 includes a housing 1210 for receiving the nipple 20K of the pacifier 10K to protect it, a cover 1220 being the plate of the pacifier 10K for covering the housing 1210, and a pivot axle 130K and biased spring 140K connected between the cover 1220 and the housing 1210 for biasing the cover 1220 and the pacifier 10K to automatically close and engage the housing 1210 with the nipple 20K contained and protected within the housing 1210. The cover 1220 and the pacifier 10K move between open and closed positions P₁ and P₂ relative to the housing 1210 so that in the closed position P₂ the housing 1210 receives and protects the nipple 20K of the pacifier 10K to keep it clean.

### Thirteenth Embodiment 1300

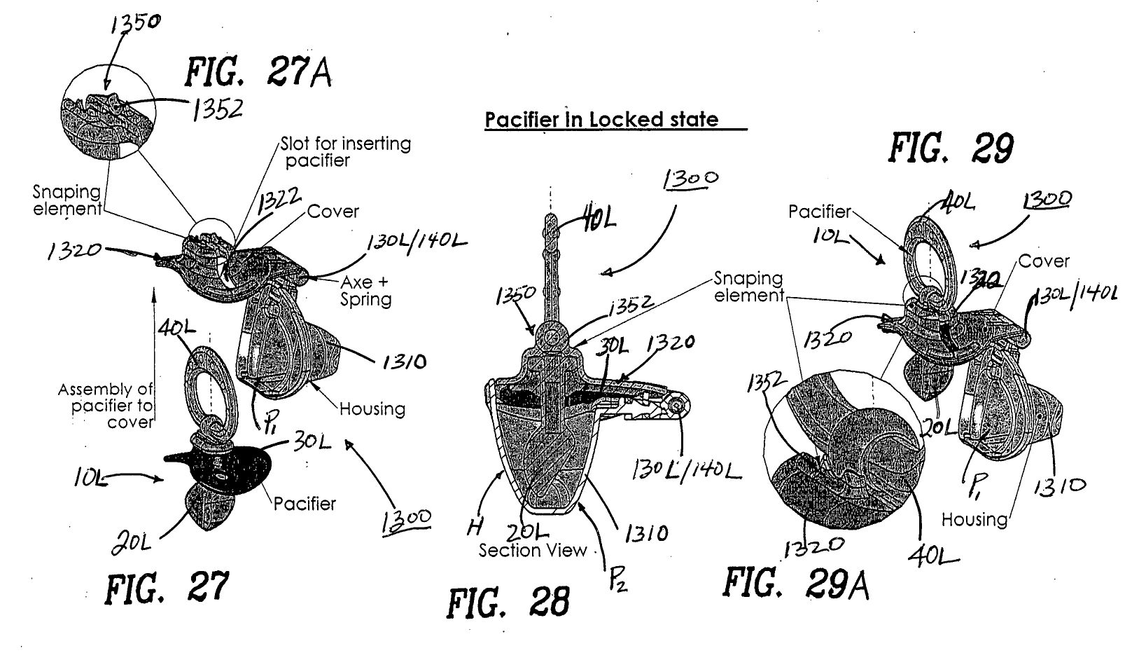

A protective assembly 1300 for receiving and protecting a pacifier 10L, as shown in FIGS. 27, 27A through 29A, wherein the pacifier 10L has a nipple 20L and a plate 30L for holding the nipple 20L. The protective assembly 1300 includes a housing 1310 for receiving the nipple 20L of the pacifier 10L to protect it, a cover 1320 for receiving and holding the plate 30L of the pacifier 10L, and a pivot axle 130L and biased spring 140L connected between the cover 1320 and the housing 1310 for biasing the cover 1320 and the pacifier 10L to automatically close and engage the housing 1310, with the nipple 20L contained and protected within the housing 1310 (see FIG. 28). Also, the cover 1320 has engaging means 1350 for engaging and holding the plate 30L of the pacifier 10L, so that the pacifier 10L moves with the cover 1320 between open and closed positions P₁ and P₂ relative to the housing 1310 so that in the closed position P₂ the housing 1310 receives and protects the nipple 20L of the pacifier 10L to keep it clean.

Additionally, the cover 1320 has a slot opening 1322 and an inner surface 1324 and the handle 40L passes through the slot opening 1322 and the plate 30L of pacifier 10L engages the inner surface 1324 of the cover 1320; and wherein the cover 1320 has engaging means 1350 in the form of snapping elements 1352, wherein the handle 40L engages the snapping elements 352 relative to the cover 1320 in order to lock the pacifier nipple 20L in its holding position H within housing 1310.

### Operation of the Present Invention

Embodiments 100, 200, 300, 400, 500, 600, 700, 800, 900, 1000, 1100, 1200 and 1300 all operate and function in a similar manner. The pacifier protective assembly 100 operates as follows: As shown in FIGS. 1 and 2, the user opens the cover 120 relative to the housing 110 via the pivot axle 130 and biased spring 140 to an opened position P₁ The user then disengages the engaging means 150 such as the bayonet locking device 152, wherein the pacifier 10 is rotated relative to the cover 120 to unlock the pacifier nipple 20 from the cover. The pacifier 10 is then removed from the opening 122 using the handle 40 of pacifier 10.

When the pacifier 10 is no longer needed by the infant, the user passes the handle 40 of pacifier 10 through opening 122 such that the plate 30 of pacifier 10 engages the inner surface 124 of cover. The cover is then manipulated using the engaging means 150 in the form of the bayonet locking device 152, where then the pacifier 10 is rotated relative to the cover 120 by the user in order to lock the pacifier nipple 20 in its holding position H within housing 110. The remaining embodiments 200 to 1300 operate in a similar manner.

### Advantages of the Present Invention

Accordingly, an advantage of the present invention is that it provides for a hinged pacifier protective assembly that hygienically protects a pacifier when not in use by an infant, baby or child.

Another advantage of the present invention is that is provides for a hinged pacifier protective assembly that protects the pacifier when the pacifier accidentally falls out of the infant's mouth and such that the pacifier is hygienically protected when hitting a floor and from being contaminated.

Another advantage of the present invention is that it provides for a hinged pacifier protective assembly that includes a cover section and a housing section being pivotally connected by a spring-biased hinge member for automatically closing the cover and housing sections together when not in use by the infant.

Another advantage of the present invention is that it provides for a hinged pacifier protective assembly that is made of flexible and durable plastic materials for easy cleaning and sanitizing for multiple re-use by a parent for their children's safety.

Another advantage of the present invention is that it provides for a hinged pacifier protective assembly that conveniently stores the pacifier within the closed cover and housing sections when not in use and prevents the pacifier from any contamination when in the closed configuration.

A further advantage of the present invention is that it provides for a hinged pacifier protective assembly that can be mass produced in an automated and economical manner and is readily affordable by parents.

A latitude of modification, change, and substitution is intended in the foregoing disclosure, and in some instances, some features of the invention will be employed without a corresponding use of other features. Accordingly, it is appropriate that the appended claims be construed broadly and in a manner consistent with the spirit and scope of the invention herein.

What is claimed:

1. A protective assembly for receiving and protecting a pacifier, wherein the pacifier has a nipple and a plate for holding the nipple, comprising:
a) said protective assembly including a housing for receiving the nipple of the pacifier to protect it, a cover for receiving and holding the plate of the pacifier, and a pivot axle and biased spring connected between said cover and said housing for biasing said cover and the pacifier to automatically close and engage said housing with the nipple contained and protected within said housing; and
b) said cover having engaging means for engaging and holding the plate of the pacifier, so that the pacifier moves with said cover between open and closed positions relative to said housing so that in said closed position said housing receives and protects the nipple of the pacifier to keep it clean.

2. A protective assembly in accordance with claim 1, wherein said cover has an opening and an inner surface and the handle of the pacifier passes through said opening, and the plate of the pacifier engages said inner surface of said cover; and wherein said cover has engaging means in the form of a bayonet locking device, wherein the pacifier is rotated relative to said cover to lock the pacifier nipple in its holding position within said housing.

3. A protective assembly in accordance with claim 1, wherein said cover has a compartment opening and an inner surface, and the plate of the pacifier engages said inner surface of said cover; and wherein said cover has engaging means in the form of locking teeth, wherein the pacifier is rotated within said compartment opening relative to said cover to lock the pacifier nipple in its holding position within said housing.

4. A protective assembly in accordance with claim 1, wherein said cover has an opening and an inner surface and the handle of the pacifier passes through said opening, and the plate of the pacifier engages said inner surface of said cover; and wherein said cover has engaging means in the form of a rubber elastic seal for engaging and sealing said cover and the plate of the pacifier within said rubber elastic seal; wherein said rubber elastic seal locks said cover and the pacifier nipple in its holding position within said housing.

5. A protective assembly in accordance with claim 1, wherein said cover has a compartment opening and an inner surface, and the handle of the pacifier is positioned within said compartment opening of said cover, and the plate of the pacifier engages said inner surface of said cover; and wherein said cover has engaging means in the form of threads, wherein the handle of the pacifier is rotated relative to said cover to lock the pacifier nipple in its holding position within said housing.

6. A protective assembly in accordance with claim 1, wherein said cover has first and second clamping sections each having an interior opening and the plate of the pacifier engages each of said interior openings of said first and second clamping sections; and wherein said cover has engaging means in the form of said clamping sections, wherein each of said clamping sections are pivotally moved in an inwardly direction in order to lock the plate and the pacifier nipple in its holding position within said housing.

7. A protective assembly in accordance with claim 1, wherein said cover has a chamber section having an interior channel opening therein and a portion of the plate engages said interior channel opening of said chamber section; and wherein said cover has engaging means in the form of said chamber section, wherein the portion of the plate is slidably received within said interior channel of said chamber section in order to lock the portion of the plate and the pacifier nipple in its holding position within said housing.

8. A protective assembly in accordance with claim 1, wherein said cover has an opening and an inner surface, the plate has a pair of opposing and spaced-apart snap mounting openings, and the handle of the pacifier passes through said opening and the plate of the pacifier engages said inner surface of said cover; and wherein said cover has engaging means in the form of a pair of opposing and spaced-apart snaps on said inner surface of said cover for detachably engaging the opposing and spaced-apart mounting openings of the plate in order to lock and hold the plate and the pacifier nipple in its holding position within said housing.

9. A protective assembly in accordance with claim 1, wherein said cover has a U-shaped opening and an inner surface and the handle of the pacifier passes through said U-shaped opening, and the plate of the pacifier engages said inner surface of said cover; and wherein said cover has engaging means in the form of a plurality of suction cups on said inner surface of said cover, wherein the plate detachably connects to said plurality of suction cups in order to lock and hold the plate and the pacifier nipple in its holding position within said housing.

10. A protective assembly in accordance with claim 1, wherein said cover has an opening, an inner surface and a pair of opposing and spaced-apart mounting openings, the plate has a pair of opposing and spaced-apart locking openings, and the handle of the pacifier passes through said opening, and the plate of the pacifier engages said inner surface of said cover such that the locking openings and said mounting openings are aligned with each other on the plate and said cover, respectively; and wherein said cover has engaging means in the form of one or more locking elements for detachably engaging the aligned locking openings and said mounting openings, respectively, in order to lock and hold the plate and the pacifier nipple in its holding position within said housing.

11. A protective assembly in accordance with claim 1, wherein said cover has an opening and an inner surface and the handle of the pacifier passes through said opening, and the plate of the pacifier engages said inner surface of said cover; and wherein said cover has engaging means in the form of a sliding locking latch for detachably engaging said cover and a portion of the handle in order to lock and hold the plate and the pacifier nipple in its holding position within said housing.

12. A protective assembly in accordance with claim 1, wherein said cover has a U-shaped opening and an inner surface and the handle of the pacifier passes through said U-shaped opening, and the plate has a plurality of loop fastener strips positioned on an exterior surface of the plate; and wherein said cover has engaging means in the form of a plurality of hook fastener strips being positioned on said inner surface of said cover, wherein the loop fastener strips on the exterior surface of the plate are matched and aligned with said hook fastener strips on said inner surface of said cover in order to lock and hold the plate and the pacifier nipple in its holding position within said housing.

13. A protective assembly in accordance with claim 1, wherein said cover has an opening and an inner surface and the handle of the pacifier passes through said opening, and the plate of the pacifier engages said inner surface of said cover; and wherein said cover has engaging means in the form of snapping elements for detachably engaging said cover and a portion of the handle in order to lock and hold the plate and the pacifier nipple in its holding position within said housing.

14. A protective assembly for receiving and protecting a pacifier, wherein the pacifier has a nipple and a plate for holding the nipple, comprising:
a) said protective assembly including a housing for receiving the nipple of the pacifier to protect it, a cover being the plate of the pacifier for covering said housing, a cover and a pivot axle and biased spring connected between said cover and said housing for biasing said cover and the pacifier to automatically close and engage said housing with the nipple contained and protected within said housing; and
b) said cover and the pacifier move between open and closed positions relative to said housing so that in said closed position said housing receives and protects the nipple of the pacifier to keep it clean.
